(19) United States
(12) Patent Application Publication
(10) Pub. No.: US 2010/0151564 A1
(43) Pub. Date: Jun. 17, 2010

(54) Biological Work Station

(76) Inventors: David J. Beebe, Monona, WI (US); Kathryn M. Haubert, Madison, WI (US); Benjamin J. Moga, Madison, WI (US)

Correspondence Address: WISCONSIN ALUMNI RESEARCH FOUNDATION, C/O BOYLE FREDRICKSON S.C, 840 North Plankinton Avenue, Milwaukee, WI, 53203, US

(21) Appl. No.: 12/333,771

(22) Filed: Dec. 12, 2008

(51) Int. Cl. C12M 1/00

(52) U.S. Cl. 435/288.7; 435/303.1

(57) ABSTRACT

A biological work station for culturing and monitoring cells includes a laminar flow bench and an incubator disposed on the bench. The bench includes a work surface adjacent the incubator that allows experiments and other manipulations to be performed within the confines of the laminar flow bench. An imaging system, such as a stereoscope, may be mounted to the bench so that images may be captured of a culture without removal of the culture from the incubator.

### FIELD OF THE INVENTION

This invention relates generally to workstations for culturing and monitoring cellular behavior and, more particularly, to a workstation having a laminar flow housing and imaging system that may capture images of a culture in the laminar flow housing without removal of the culture from the laminar flow housing. In one exemplary implementation, the workstation further includes an incubator and work surface adjacent the incubator such that experiments and the like may be performed on a culture without removal of the culture from the workstation and the controlled environment of the laminar flow housing.

### BACKGROUND AND SUMMARY OF THE INVENTION

Cells, such as stem cells, bacteria, fungi, and other cellular material, are commonly cultured in incubators that provide a temperature and humidity controlled environment in which cells may be grown and the stages of cellular growth monitored. As the cellular material matriculates through its stages of growth, it is often desirable to capture images of the growth process. To this end, bio-stations, such as the BioStationCT commercially available from Nikon Instruments, allows computed tomography (CT) images to be acquired during the cellular growth process while maintaining the cultures in their optimized, and closed environment.

One of the drawbacks of conventional biological workstations, including those having integrated imaging devices, is that the controlled environment of the incubator is exposed when cultures are added or removed from the incubator. This is particularly problematic when dealing with microfluidic devices that are used with human and animal in-vitro fertilization (IVF). That is, one of the challenges in working with embryos or cells in microfluidic devices is the effect of “opening the door” when the devices are moved to and from the incubator for manipulation and analysis. It has been found that the effects of the humidity and temperature changes caused by such movement can have a significant impact on the cells and the developing embryos.

The present invention is directed to a work station well-suited for culturing and monitoring cellular material that overcomes the aforementioned drawbacks. In one embodiment, the present invention includes an incubator and a work area generally adjacent the work area; both of which are contained within a laminar flow system that provides a clean and controlled environment for working with the cellular material. In a further embodiment, an imaging system is associated with the laminar flow system that allows images to be acquired of the cellular material without removal of the cellular material from the incubator. In yet another embodiment, the incubator has a turn-table or carousel capable of holding various types of culture devices, such as dishes, tubes, slides, and the like. The carousel may be rotated by a suitable drive motor to selectively place a culture or cellular sample to an imaging position that is in the field of view of the imaging system. In yet a further embodiment, the incubator includes a water bath that provides temperature and evaporation control of the conditions inside the incubator. In one embodiment, the water bath is maintained at a level sufficient to partially submerge the culture devices.

It is therefore an object of the present invention to provide a bio-station equipped to provide incubation of cellular material, imaging of the cellular material without removal of the cellular material from the bio-station, and a climate controlled work area that allows researchers and scientists to carry out various tasks associated with the cellular material, such as inspection under a microscope and experiments without removal of the cellular material from the controlled environment provided by a laminar flow system.

It is a further object of the present invention to provide a laminar flow system having an incubator and a temperature controlled work area generally adjacent the incubator.

It is yet another object of the present invention to provide an integrated laminar flow system and an imaging system that can capture images of cellular material without removal from the laminar flow system. In accordance with yet a further object of the invention, the laminar flow system includes an incubator that provides a climate controlled environment for cellular growth and the imaging system is capable of capturing images of the cellular material without removal of the cellular material from the incubator.

Therefore, in accordance with one aspect of the invention, an apparatus is provided that includes a laminar flow hood and a table connected to the laminar flow hood. The table includes an opening and an incubator is supported on the table. The incubator includes a housing and a media support disposed in the housing, with a portion of the housing seated above the opening formed in the table. An imaging device is aligned with the opening formed in the table and is configured to acquire images of a media sample supported by the media support without removal of the media from the incubator.

Other aspects, features, and advantages of the invention will become apparent to those skilled in the art from the following detailed description and accompanying drawings. It should be understood, however, that the detailed description and specific examples, while indicating preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is an isometric view of a working station having a work surface, an incubator disposed on the work surface, and an imaging system for acquiring images of cellular material contained within the incubator according to one embodiment of the present invention;

FIG. 2 is an isometric view of the incubator shown disposed on the work surface;

FIG. 3 is an exploded view of the incubator of FIG. 2;

FIG. 4 is an isometric view of the imaging system of FIG. 1;

FIG. 5 is a section view of the incubator and work surface taken along line 5-5 of FIG. 2;

FIG. 6 is a section view of the incubator taken along line 6-6 of FIG. 5;

FIG. 7 is an end elevation view of the incubator and work surface shown in FIG. 5;

FIG. 8 is a section view of the incubator and work surface taken along line 8-8 of FIG. 5; and

FIG. 9 is a section view of the incubator and work surface similar to that shown in FIG. 8 with an access door of the incubator shown in an open position.

### DETAILED DESCRIPTION OF THE DRAWINGS

The present invention is directed to a work station particularly well-suited for culturing, monitoring, imaging, and working with human, animal, or plant cellular material, such as stem cells, tissue, bacteria, fungi, and embryos. It is understood however that the invention is not limited to use with biological samples and cultures.

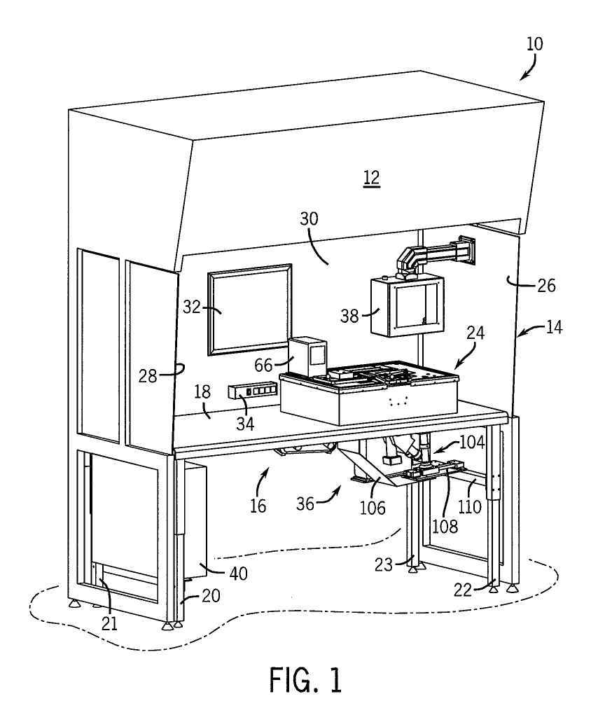

FIG. 1 shows a laminar flow system 10 having a laminar flow hood 12 providing a housing for a high efficiency particulate air (HEPA) filter (not shown). As known in the art, laminar flow systems, such as laminar flow benches, are commonly used in biomedical research to maintain a clean environment for cells, bacteria, fungi, and tissues, hereinafter referred to as “cellular material”, and thereby prevent contamination of the cellular material. The hood 12 is supported by a frame 14 that generally provides an enclosure for a work table 16 that has a work surface 18 supported by legs 20, 21, 22, and 23. An incubator 24 is positioned on the work surface 18, which as will be further described below, provides a temperature and humidity controlled environment for cellular growth. In one embodiment, heating elements (not shown) are embedded in the work surface 18, or at least a portion thereof adjacent the incubator 24, to heat the work surface 18. Heating the work surface 18 reduces the differences in temperature between the incubator 24 and the work surface 18.

The frame 14 includes a pair of side panels 26, 28 connected to one another by a transversely oriented back panel 30. A computer monitor 32 is mounted to the back panel 30 as is a power strip 34, to which various electronic devices may be connected, such as a notebook computer, microscope, or other equipment.

An imaging system 36, which in one embodiment is a stereoscope, is mounted to the work table 16 and may be used to acquire images of cellular material disposed in the incubator 24. Thus, images may be captured of the cellular material without removal of the cellular material from the incubator 24. The acquired images may be recorded and stored as known in the art or displayed on a viewer 38 that is mounted to side panel 26.

In one embodiment, the incubator 24 and the imaging system 36 are controlled by a computer 40 mounted to legs 20, 21. In this regard, a user, such as a researcher or scientist, may make inputs to the computer 40 using conventional user input tools (not shown) and the computer 40, which executes appropriate software, controls operation of the incubator 24 and the imaging system 36 accordingly. It is recognized that the inputs for controlling operation of the incubator 24 and the imaging system 36 may be made remotely from the work station 10 using conventional remote connectivity and communication hardware and software.

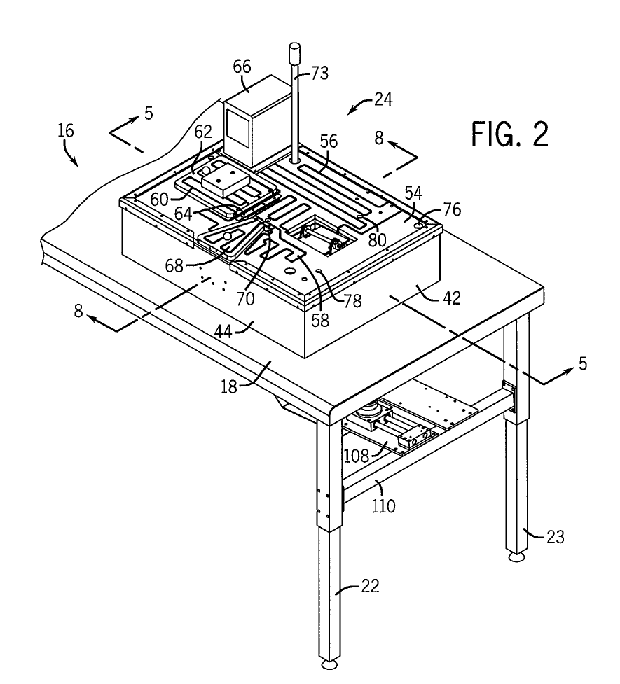

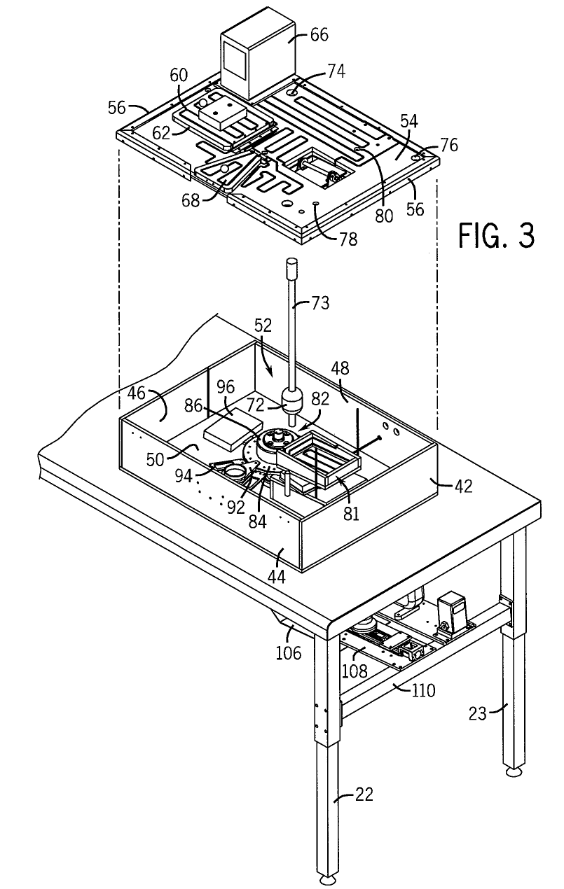

Turning now to FIGS. 2-3, the incubator 24 is defined by a set of sidewalls 42, 44, 46, 48 extending upward from a base panel 50 that rests atop the work surface 18. The sidewalls 42, 44, 46, 48, and the base panel 50 may be integrally formed with one another or connected via gaskets or similar devices, and collectively define an incubation volume 52 that is closed by a cover 54. The cover 54 is secured to the sidewalls 42, 44, 46, 48 by brackets 56 positioned along an outer edge of the cover 54. In a preferred embodiment, the sidewalls, base panel, and cover are formed of a transparent synthetic material, but it is recognized that other materials may be used.

As noted above, the sidewalls 42-48 and the base panel 50 are sealingly coupled to one another, or alternately, integrally formed as a single unit, such that a water bath may be maintained in the incubator 24. The water bath may be heated by a series of heating elements 56, 58, 60. In one embodiment, the heating elements 56, 58 are defined within the top cover 54 whereas heating element 60 is defined within an access door 62 that is connected to the top cover 54 by a hinged connection 64. A heater control 66 is mounted to the top cover 54 and regulates the heat output of the heating elements 60, as known in the art, to provide a desired temperature within the incubator and control against condensation being formed on the underside of the top cover 54.

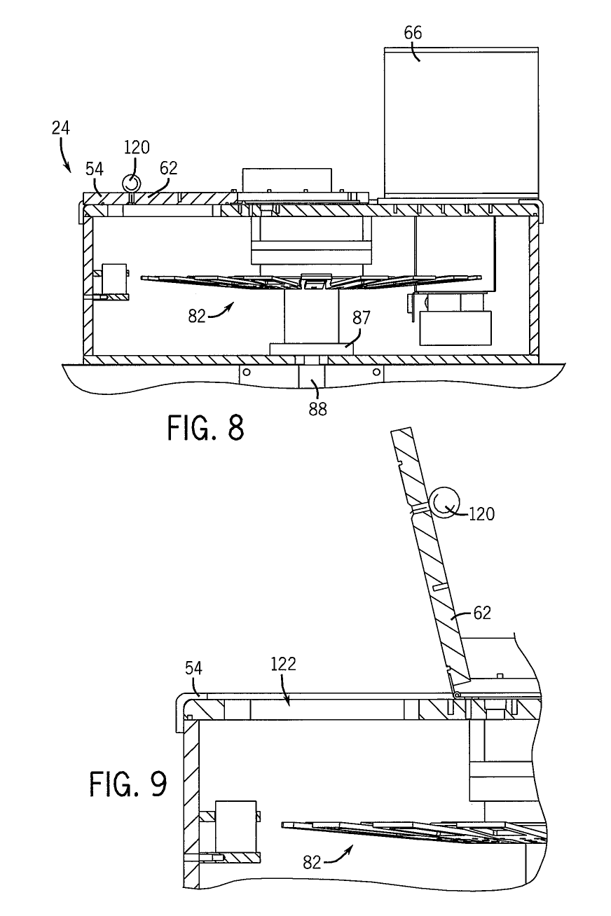

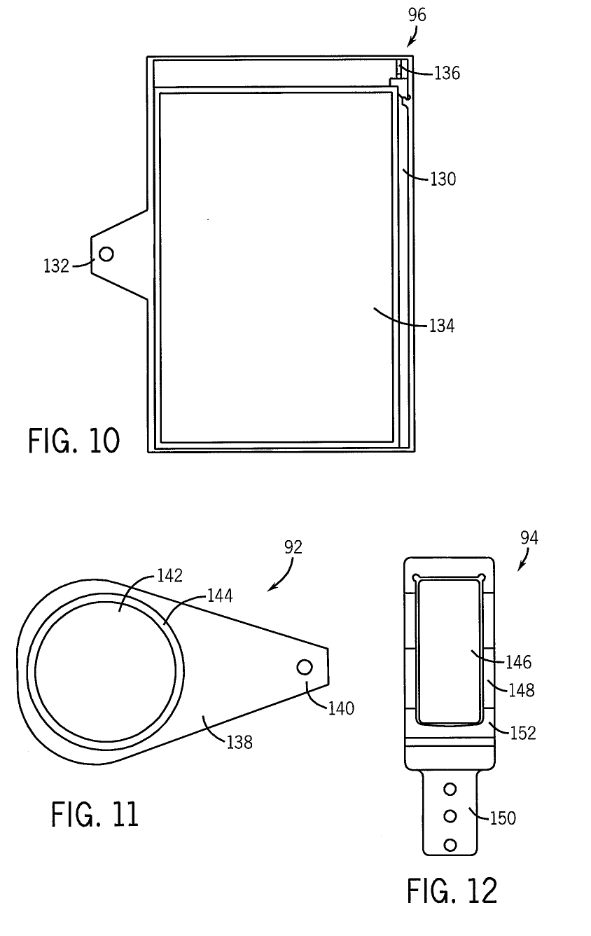

In addition to access door 62, a generally triangularly shaped access door 68 is coupled to the top cover 54 by a hinged connection 70. Access door 62 may be opened for loading large culture dishes into the incubator, as shown in FIGS. 8-9, whereas door 68 may be used load smaller culture dishes, such as slides, into the incubator as well as performing pipetting activities.

The water level of the aforementioned water bath is detected by a buoy or float 72 that is supported on a rod 73 extends through an opening 74 in the top cover 54. In a preferred embodiment, the water level information is used to automatically add water to the incubator from a water supply (not shown) as water evaporates and thus drops the water level. Gas is supplied to the incubator via gas hoses (not shown) passing through openings 76, 78, and 80 formed in the top cover 54.

The cover 54 further includes a generally square shaped housing 81 that provides an enclosure for a light (not shown) that is designed to provide backlighting for images acquired by the imaging system 36.

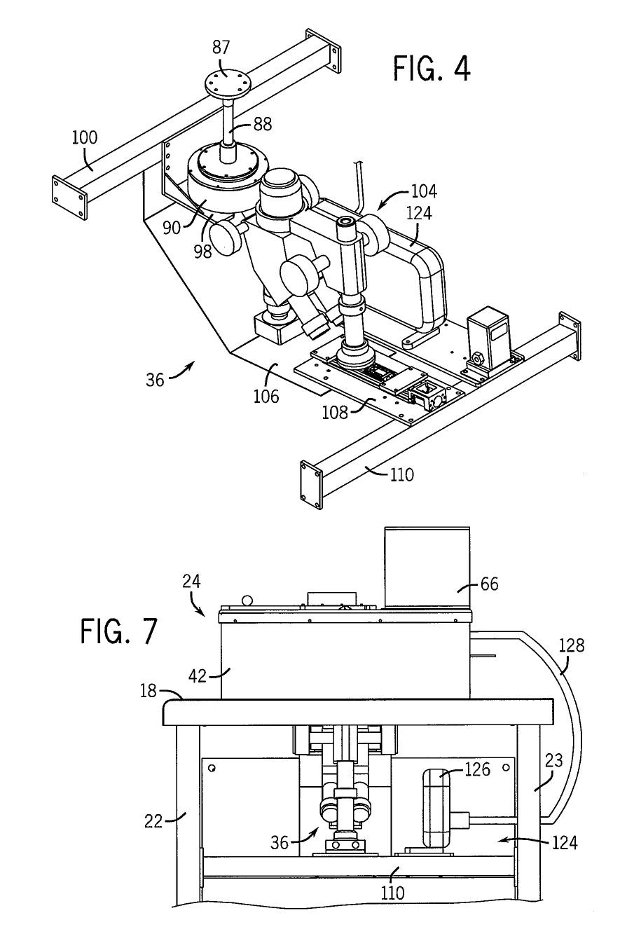

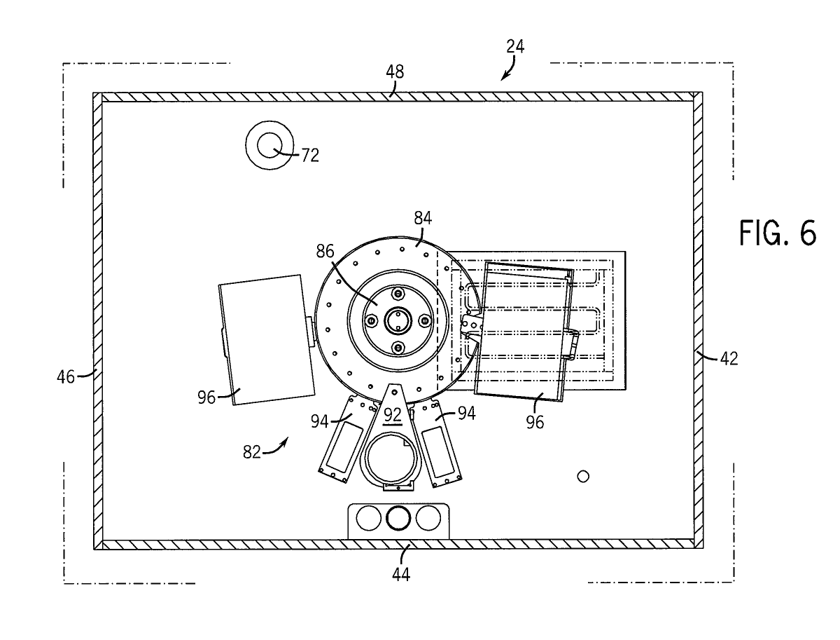

With additional reference to FIGS. 4 and 6, the incubator 24 further includes a loader 82, that in a preferred embodiment, includes a carousel having a disc 84 coupled to a hub 86 having a drive plate 87 that is connected to a shaft 88 that is driven by a drive motor 90. Various culture device holders may be fastened to the disc 84, such as a Petri dish holder 92, a slide holder 94, and a tray 96. It is understood that other types of holders may be attached to the disc, such as holders designed to hold vials and test tubes. In a preferred embodiment, the device holders are attached to the disc in a manner that allows the device holders to be easily attached and detached from the loader to accommodate variances in the types of cultures placed in the incubator 24.

As further shown in FIG. 4, the drive motor 90 is supported by a bracket 98 that is fastened to a crossbar 100 that spans the width of the work table 16 and is fastened to a downwardly extending lip 102 of the work surface 18. Also mounted beneath the work surface 18 is the imaging system 16 that generally includes a stereoscope 104, which are known in the art for capturing images of cellular matter, such as stem cells. The stereoscope 104 is supported by a platform 106 that is coupled to the crossbar 100 and a base 108 that is connected to the platform and crossbar 110 that extends between legs 22, 23 of the work table 16. Operation of stereoscopes is known in the art and thus will be not be described in further detail herein; however, it is noted that a stereoscope represents one type of imaging device that may be incorporated into the work station 10 and, as such, it is contemplated that other types imaging devices may be used.

Referring now to FIG. 7, as noted above, a water bath is maintained in the incubator 24 to provide temperature and humidity control within the incubator 24. Preferably, the water bath is sterilized by circulating the water through a sterilizer 124, that in one embodiment, includes a UV light source (not shown) mounted in a housing 126 generally adjacent the imaging system 36. As the UV light may be damaging to the cellular material, the water is circulated to the sterilizer 124 via supply and return flow paths (not shown) contained in hose 128.

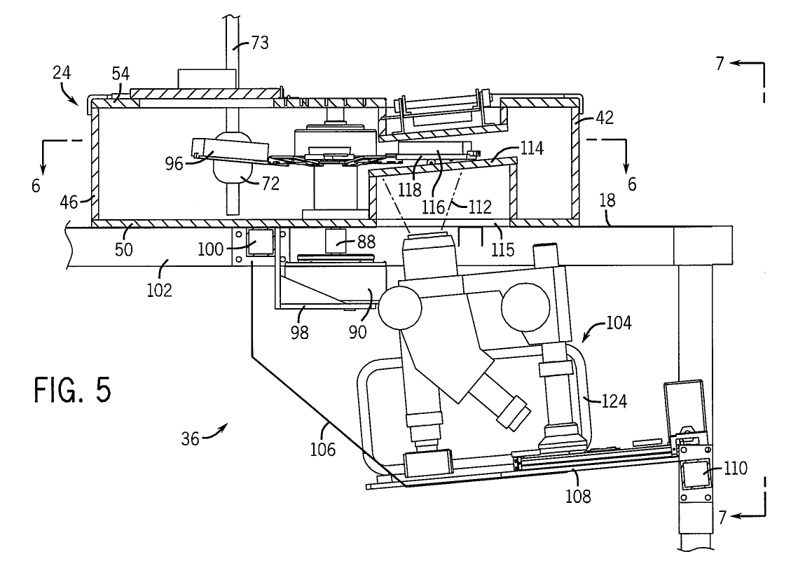

The stereoscope 104 is mounted such that the field-of-view (FOV) 112 of the stereoscope falls within an imaging window 114 defined in the base 50 of the incubator 24. As shown in FIG. 4, the imaging window 114 lies generally above an opening 115 formed in the work surface 18 of the work table 16. The imaging window 114 is defined such that the stereoscope 104 may capture images of culture in a device held in a device holder that is placed above the imaging window 114. Thus, in the example shown in FIG. 5, the stereoscope 104 may capture images of a culture contained in device 116 held by device holder 118. As described above, the various cultures may be rotated to an imaging position via rotation of the carousel 82.

Referring again to FIG. 8-9, the incubator 24 is constructed to have an access door 62 that can be selectively opened by pulling upward on handle 120. When the access door 62 is in the open position, FIG. 8, a user may place or remove items from the loader through opening 122 in the top cover 54. The opening 122 is sized such that a user can load a large culture dish, such as tray 96, onto the loader.

From the foregoing description, it will be appreciated that the present invention provides a work station at which cellular growth may be grown, monitored, imaged, and manipulated, e.g., experiments performed thereupon, in a temperature and humidity controlled environment. The partial submersion of the culture devices provides temperature control as well as evaporation control such that a relatively constant condition is maintained in the incubator. In a preferred embodiment, the water bath is sterilized by a UV system having a UV light, as described above.

Various modes of carrying out the invention are contemplated as being within the scope of the following claims particularly pointing out and distinctly claiming the subject matter, which is regarded as the invention.

1. An apparatus comprising:
a laminar flow hood;
a table connected to the laminar flow hood and having an opening formed therein;
an incubator supported on the table and having a housing and a media support disposed in the housing, a portion of the housing seated above the opening formed in the table; and
an imaging device aligned with the opening formed in the table and configured to acquire images of a media sample supported by the media support without removal of the media from the incubator.

2. The apparatus of claim 1 wherein the media support is configured to support multiple media samples.

3. The apparatus of claim 2 wherein the media support includes a carousel coupled to a drive that rotates the carousel to position a media sample at a desired position in the housing.

4. The apparatus of claim 3 wherein the carousel includes a hub coupled to the drive, a disc coupled to the hub, and a series of radially spaced platforms coupled to and extending from the disc.

5. The apparatus of claim 3 wherein each platform has a first end and a second end, the first end coupled to the disc and wherein the second end is raised relative to the first end.

6. The apparatus of claim 4 wherein at least one of the platforms is configured to hold a Petri dish.

7. The apparatus of claim 4 wherein at least one of the platforms is configured to hold a slide.

8. The apparatus of claim 1 wherein the imaging device includes a stereoscope.

9. The apparatus of claim 1 further comprising fluid disposed in the housing.

10. The apparatus of claim 9 further comprising a heater assembly configured to maintain a temperature of the fluid in the housing at a desired temperature.

11. A system comprising:
a laminar flow station;
an incubator disposed in the laminar flow station; and
a work area adjacent the incubator and disposed in the laminar flow station.

12. The system of claim 11 wherein the incubator includes a loader configured to support a number of pods, each pod capable of holding a sample.

13. The system of claim 12 further comprising an imaging device adjacent the incubator and configured to acquire an image of a sample disposed on a pod without removal of the sample from the incubator.

14. The system of claim 11 further comprising a table having an opening therein and wherein the incubator is positioned on a top surface of the table with a portion of the incubator over the opening and wherein the imaging device is mounted to the table such that the imaging device sits below a bottom surface of the table and acquires an image of the sample through the opening in the table.

15. The system of claim 14 wherein the table includes a work surface adjacent the incubator.

16. The system of claim 11 wherein the loader includes a disc having an inner radial end coupled to a drive motor configured to rotate the disc to position a sample in a desired position within the incubator.

17. The system of claim 16 wherein the pods are removably coupled to an outer radial end of the disc.

18. The system of claim 11 wherein the pods include at least one of a Petri dish pod configured to hold a Petri dish or a slide pod configured to hold a slide.

19. The system of claim 11 wherein the incubator includes a water bath and wherein the pods are partially submerged in the water bath.

20. The system of claim 19 further comprising a UV source that sterilizes the water bath.

21. The system of claim 11 further comprising a monitor proximate the incubator and including a screen onto which images acquired by the imaging device are displayed.

22. An incubator comprising:
a carousel capable of being rotated by a drive motor; and
a plurality of culture device holders removably coupled to the carousel.

23. The incubator of claim 22 wherein the plurality of device holders includes device holders for holding Petri dishes.

24. The incubator of claim 22 wherein the plurality of device holders includes device holders for holding slides.

25. The incubator of claim 22 wherein the plurality of device holders includes device holders for holding trays.
